(19) United States
(12) Patent Application Publication
(10) Pub. No.: US 2015/0086418 A1
(43) Pub. Date: Mar. 26, 2015

(54) FOCUSED AIRFLOW IN A TWO TRUCK WIDE VIVARIUM

(71) Applicant: SPX CORPORATION, Charlotte, NC (US)

(72) Inventors: Gary C. Snyder, Montoursville, PA (US); David H. Gibson, Lewisburg, PA (US)

(21) Appl. No.: 14/038,237

(22) Filed: Sep. 26, 2013

(51) Int. Cl. A61L 2/06

(52) U.S. Cl. CPC A61L 2/06
USPC 422/22; 422/243

(57) ABSTRACT

A sterilization system including an air intake and an air exhaust fluidly connected to a conditioning plenum is provided. A heater is arranged inside the conditioning plenum downstream from the air intake. The system includes a first sterilization chamber adjacent to a second sterilization chamber, a first pressure plenum adjacent to the first sterilization chamber and fluidly connected to the first sterilization chamber and the conditioning plenum and a second pressure plenum adjacent to the second sterilization chamber and fluidly connected to the second sterilization chamber and the conditioning plenum is provided. The system further includes a first return duct arranged between the first and second sterilization chambers and fluidly connected to the first sterilization chamber and the conditioning plenum and a second return duct arranged between the first and second sterilization chambers and fluidly connected to the second sterilization chamber and the conditioning plenum.

### BACKGROUND OF THE DISCLOSURE

### Field of the Disclosure

The present invention relates generally to a sterilization system. More particularly, the present invention relates to a sterilization system using dry heat.

Traditionally, steam is a common way to sterilize cages and other contaminants. A widely-used device for heat sterilization is the autoclave. Autoclave commonly use steam heated to 121° C. (250° F.), at 103 kPa (15 psi) above atmospheric pressure to transfer sufficient heat to sterilize the content. For effective autoclaving, the steam needs to be able to penetrate the entire device. For this reason, an autoclave must not be overcrowded, and the lids of bottles and containers must be ajar. Furthermore, indicators must be placed in the most difficult place to sterilize to ensure that steam actually penetrates these areas.

Unfortunately, the use of steam autoclaves bears high initial cost, high operating cost (steam boiler, distribution lines, high volumes or water, and licensing of operators), and high maintenance costs. Furthermore, the user of the steam autoclaves must allow the steam to cool down to ambient liquid form before disposing to the drain.

Accordingly, it is often more desirable to use a sterilization system that uses dry heat convection rather than an autoclave that uses steam. The use of a dry heat convection sterilizer eliminates the high initial costs, operating costs and maintenance costs of sterilization via the autoclave. Furthermore, the use of dry heat convection sterilizer benefits the environment because hot steam will not be released from the system into the environment.

Although dry heat sterilizers have many advantages over using steam autoclaves, current batch cycle times are longer for dry heat sterilizers than the cycle times required in an autoclave. Furthermore, most sterilizers today must accommodate multiple units of animal caging systems in which two truck units are loaded side by side within the sterilizer. This two-truck load arrangement often requires an extended cycle time using dry heat for sterilization because cold spots develop on one side of the system or because the air inlet side heats up faster than the other side.

Therefore, it would be advantageous to have a dry heat convection sterilizer that has reduced batch cycle times and improved heating efficiency in particular for sterilization systems that use a two truck wide load arrangement. Reduced cycle time per batch would allow a greater number of batches over a specific period of time and also reduced utility costs per batch.

### SUMMARY OF THE DISCLOSURE

According to an aspect of the disclosure, a sterilization system including an air intake and an air exhaust fluidly connected to a conditioning plenum is provided. A heater is arranged inside the conditioning plenum downstream from the air intake. The system includes a first sterilization chamber adjacent to a second sterilization chamber, a first pressure plenum adjacent to the first sterilization chamber and fluidly connected to the first sterilization chamber and the conditioning plenum and a second pressure plenum adjacent to the second sterilization chamber and fluidly connected to the second sterilization chamber and the conditioning plenum is provided. The system further includes a first return duct arranged between the first and second sterilization chambers and fluidly connected to the first sterilization chamber and the conditioning plenum and a second return duct arranged between the first and second sterilization chambers and fluidly connected to the second sterilization chamber and the conditioning plenum.

According to another aspect of the disclosure, a method of sterilizing animal cages is provided. The method includes (a) providing a sterilization system including an air intake and an air exhaust fluidly connected to a conditioning plenum; a heater arranged inside the conditioning plenum downstream from the air intake; a first sterilization chamber adjacent to a second sterilization chamber; a first pressure plenum adjacent to the first sterilization chamber and fluidly connected to the first sterilization chamber and the conditioning plenum; a second pressure plenum adjacent to the second sterilization chamber and fluidly connected to the second sterilization chamber and the conditioning plenum; a first return duct arranged between the first and second sterilization chambers and fluidly connected to the first sterilization chamber and the conditioning plenum; and a second return duct arranged between the first and second sterilization chambers and fluidly connected to the second sterilization chamber and the conditioning plenum. The method further includes (b) placing containers of animal cages to be sterilized in the first and second sterilization chambers, (c) directing heated air from the conditioning plenum to the first pressure plenum and from the first pressure plenum to the first sterilization chamber and from the first sterilization chamber through the first return duct; (d) directing heated air from the conditioning plenum to the second pressure plenum and from the second pressure plenum to the second sterilization chamber and from the second sterilization chamber through the second return duct; (e) returning the air from the first return duct to the conditioning plenum and (f) returning the air from the second return duct to the conditioning plenum.

Additional features, advantages, and embodiments of the disclosure may be set forth or apparent from consideration of the following detailed description, drawings, and claims. Moreover, it is to be understood that both the foregoing summary of the disclosure and the following detailed description are exemplary and intended to provide further explanation without limiting the scope of the disclosure as claimed.

### BRIEF DESCRIPTION OF THE DRAWINGS

The accompanying drawings, which are included to provide a further understanding of the disclosure, are incorporated in and constitute a part of this specification, illustrate embodiments of the disclosure and together with the detailed description serve to explain the principles of the disclosure. No attempt is made to show structural details of the disclosure in more detail than may be necessary for a fundamental understanding of the disclosure and the various ways in which it may be practiced. In the drawings:

FIG. 1 is a front view illustrating a sterilization system according to an embodiment of the invention.

FIG. 2 is a sectional view of the front of the sterilization system of FIG. 1 showing airflow.

FIG. 3 is a side view of the sterilization system of FIG. 1.

FIG. 4 is a sectional side view of the sterilization system of FIG. 1.

FIG. 5 is a detail view of the top view of the sterilization system of FIG. 1.

### DETAILED DESCRIPTION OF THE DISCLOSURE

The embodiments of the disclosure and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the disclosure. The examples used herein are intended merely to facilitate an understanding of ways in which the disclosure may be practiced and to further enable those of skill in the art to practice the embodiments of the disclosure. Accordingly, the examples and embodiments herein should not be construed as limiting the scope of the disclosure, which is defined solely by the appended claims and applicable law. Moreover, it is noted that like reference numerals represent similar parts throughout the several views of the drawings.

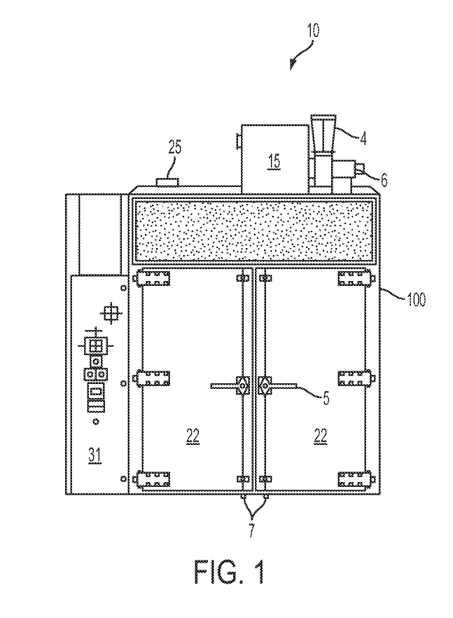

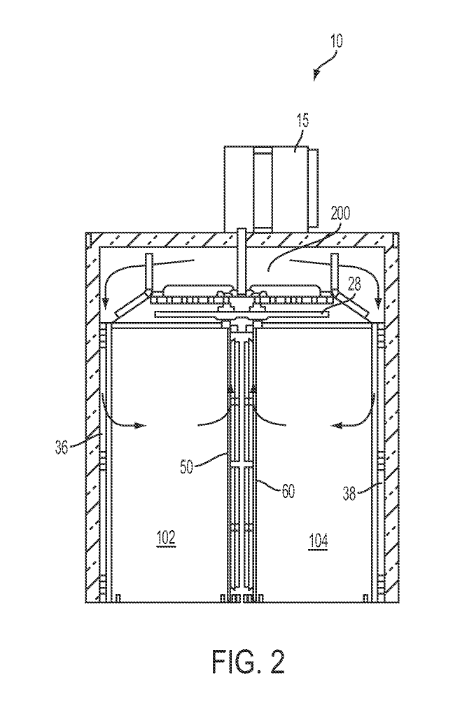

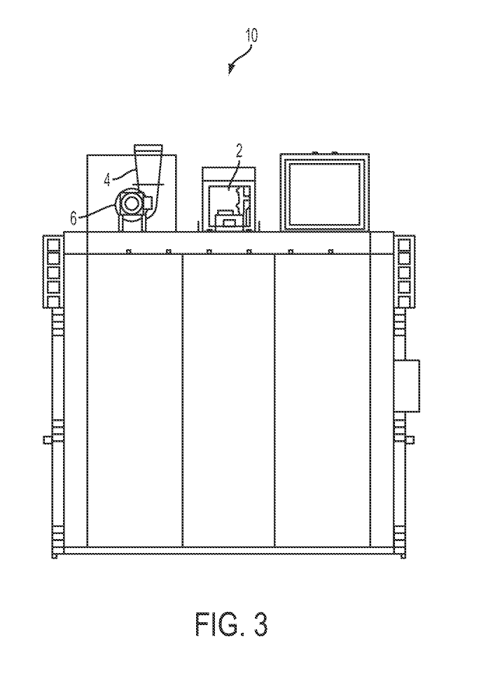

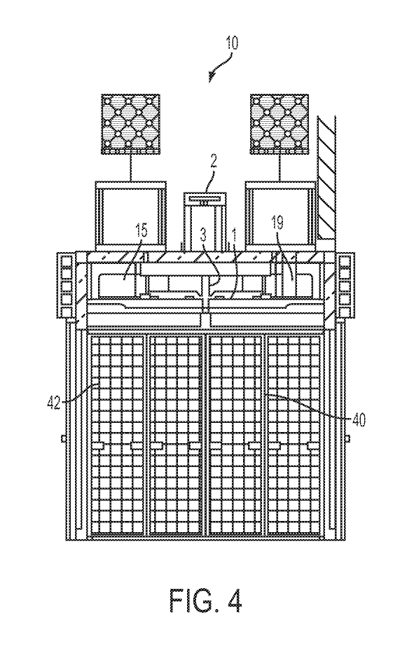

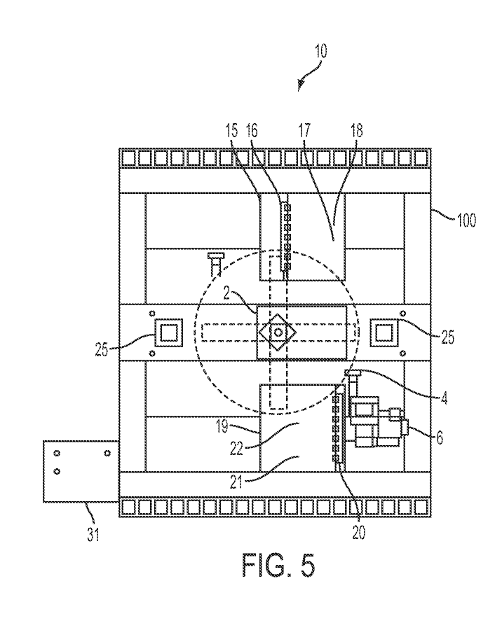

FIG. 1 shows a front view illustrating a sterilization system 10 according to an embodiment of the invention; FIG. 2 is a sectional view of the front of the sterilization system of FIG. 1 showing the airflow pattern; FIG. 3 is a side view of the sterilization system of FIG. 1; FIG. 4 is a sectional side view of the sterilization system of FIG. 1; and FIG. 5 is a detail view of the top view of the sterilization system of FIG. 1.

As shown in FIG. 1, the sterilization system 10 includes a housing 100, an intake filter box 15, an exhaust filter box 19 (FIG. 4), an exhaust blower 4, an exhaust blower motor 6 (FIG. 3), an electrical junction box 25, a recirculating motor 2 (FIG. 5), doors 22 with latches 7, a handle 5, and a control console 31.

The exhaust blower 4, the intake filter box 15, the exhaust filter box 19, the electrical junction box 25, the recirculating fan motor 52 and the recirculating motor 2 may be mounted on top of the housing 100.

The housing 100 shown is generally rectangular shaped. Although rectangular, it should be understood that the housing 100, and thus the sterilization system 10, may conceivably be a different overall shape if necessary to suit the requirements imposed by the environment in which the system 10 is used.

The control console 31 has controllers and recorders for controlling and recording the temperature of the sterilization cycle during operation of the system 10. The control console 31 also has various buttons and indicators for controlling the sterilization systems such as, for example, start button, stop button, fault indicator, temperature display, timer, alarm, etc.

In the present embodiment, the sterilization system 10 has two identical filters located within the intake filter box 15 and the exhaust filter box 19 (FIG. 4). An intake filter cleans air as the air is directed into the chamber from the atmosphere, while the exhaust filter ensures that containments in the chamber is contained in the exhaust filter box 19 and not released into the atmosphere when the hot air is vented. Another function of the filters is to prevent ambient air from entering the chamber through the exhaust outlet to re-contaminate the contents when the sterilization process is complete. Furthermore, the exhaust blower 4 is attached to the exhaust filter box 19 and pulls air into and through the intake filter box and through the conditioning plenum 200 and pulls exhaust air into the atmosphere away from the housing 100. The exhaust blower 4 is attached to an exhaust blower motor 6 as shown in FIG. 3.

As indicated, the intake filter box 15 and the exhaust filter box 19 each contains high efficiency filters including, for example, HEPA filters. Furthermore, ports 16, 17, 18 on the intake filter box 15 are used to introduce test materials to test the integrity of the intake filter box 15. Similarly, exhaust filter box 19 (FIG. 5) also has ports 20, 21, 22 for similar purposes. The ports 16, 17,18, 20, 21, 22 check the upstream and downstream of the airflow of the filters to ensure that the filter box 15, 19 is functioning properly. For example, an operator can confirm that there is no leakage in the system or any obstruction of airflow in the filter. There is a set of three ports: an intake input port, an intake scan port and an intake 100% test port. In operation, a test material is introduced through the intake input port, and a sensor in the port will verify the concentration of the material. Then, another probe is placed in the intake scan port, which is downstream to the intake input port, to measure the concentration of the material that passed through the filter. If the filter scan test failed, an attempt is made to correct the problem. For example, the operator can increase the clamp force on the filter gasket or seal any small leak in the filter media with silicon caulk. If these measures are not successful, a new filter will be installed.

In another embodiment, the sterilization system 10 can have one or more filters depending on the user's specification and application. In situations where the users are located in a class 100 atmosphere, an intake filter might not be necessary. In that situation, the production cost of manufacturing the sterilization system will decrease. Nonetheless, an exhaust filter can prevent ambient air from re-entering and contaminating the chambers 102, 104 (FIG. 2).

As shown in FIG. 2, the sterilization system 10 has two distinct sterilization chambers: a first sterilization chamber 102 and a second sterilization chamber 104 and a conditioning plenum 200. The first and second chambers 102, 104 are adjacent to each other and separated by a first return duct 50 and a second return duct 60. The return ducts 50, 60 are also adjacent to each other. Each one of the chambers 102, 104 has a dedicated return duct 50, 60. For example, the first sterilization chamber 102 has a first return duct 50. The second sterilization chamber 104 has a second return duct 60.

The conditioning plenum 200 is arranged above both chambers 102, 104. The chambers 102, 104, and the conditioning plenum 200 are located within the inside of the housing 100 of the sterilization system 10. The conditioning plenum 200 is fluidly connected to the first return duct 50 and the second return duct 60. The first return duct 50 is fluidly connected to the first chamber 102 and the second return duct is fluidly connected to the second chamber 104.

The sterilization system 10 has a recirculating fan 1, a recirculating motor 2, a fan shaft 3, and heaters 28. The recirculating fan 1 and the heaters 28 are located within the conditioning plenum 200. The recirculating fan 1 is situated above the heaters 28 inside the conditioning plenum 200 as shown in FIG. 2. The recirculating fan 1 is used to circulate the heated air as discussed in detail below. The recirculating fan 1 is connected to a fan shaft 3 which extends vertically through the conditioning plenum 200 and is connected to the recirculating motor 2. The recirculating motor 2 is mounted on the housing 100 above the plenum 200.

The system 10 intakes air through the intake filter box 15 and into the conditioning plenum 200. Air returns from the first chamber 102 and from the second chamber 104 to the conditioning plenum 200 via the first and second return ducts 50, 60, respectively. The air returning from the sterilization chambers 102, 104 is received by the conditioning plenum 200 and is heated as it passes through the heaters 28. Once the air is heated, it is then circulated by the recirculating fan 1.

The conditioning plenum 200 is fluidly connected to a first pressure plenum 36 and a second pressure plenum 38. Air must flow from the conditioning plenum 200 to the first pressure plenum 36 and the second pressure plenum 38 where it reaches the first and second chambers 102, 104. The first chamber is 102 is fluidly connected to the first pressure plenum 36. The second chamber 104 is fluidly connected to a second pressure plenum 38. The air is directed to pass horizontally through the first chamber 102 until it reaches the first return duct 50 and returns to the conditioning plenum 200 where it is reheated and re-circulated. Air is also directed to pass horizontally through the second chamber 104 until it reaches the second return duct 60 and returns to the conditioning plenum 200 where it is reheated and re-circulated. The horizontal flow through the chambers 102, 104 is described in further detail below.

FIG. 4 illustrates a sectional side view of the sterilization system 10. The sterilization system 10 has a set of semi-pierced duct walls 40 inside the first and second chambers 102, 104 as shown in FIG. 4. The semi-pierced duct walls 40 have numerous adjustable diffuser panels 42. In operation, the diffuser panels 42 are angled in such a way that they aid the airflow in and out of the chamber 102, 104. These diffuser panels 42 are adjustable according to the needs of the user and the contents being sterilized in the system.

During operation of the sterilization system 10, hot air travels down the first and second pressure plenums 36, 38 where the hot air will enter the first and second chambers 102, 104 through the diffuser panels 42 of each chamber's respective semi-pierced duct walls 40. Hot air then exits the chambers 102, 104 through their respective return ducts 50, 60. The air is re-heated at the heaters 28, and is re-circulated by the fan 3 within the sterilization system 10.

As discussed above, some systems 10 can contain multiple filter boxes if necessary. As shown in FIG. 4, an intake filter box 14 and exhaust filter box 19 are mounted on top of the housing 200 of the sterilization system 10 and exhaust airflow blower 4. A single heater or a set of heaters 28 may be are situated inside the conditioning plenum 200. The heaters 28 are connected to the electrical junction box 25, each with electrical communication with airflow switches (not shown).

FIG. 2 illustrates the airflow path when the sterilization system 10 is in operation. Arrows are shown in FIG. 2 to illustrate airflow. Airflow enters and exits through the intake filter box 14 and the exhaust filter box 19 of the sterilization system 10, respectively. Air is heated while moving past the heaters 28 and circulated by the recirculating fan 3. Heated air is directed from the conditioning plenum 200 down to the first pressure plenum 36 and down the second pressure plenum 38.

Hot air traveling down the first pressure plenum 36 enters the first chamber 102, which directs the hot air across the first chamber 102. Once inside the chamber 102, the hot air heats up the internal area of the first chamber 102 by flowing horizontally from left to right across the chamber 102 and exits the first chamber 102 via the first return duct 50. Once exited from the first chamber 102, the hot air is re-heated at the heater 28, and is re-circulated within the sterilization system 10. In a similar fashion, air flows from the second pressure plenum 38 horizontally from right to left across the second chamber 104 and to the second return duct 60. The air then reaches the second return duct 60 and flows back to the conditioning plenum 200 where it is reheated and re-circulated.

One key advantage of the present invention is that the sterilization system 10 uses a separate airflow path for each sterilization chamber 102, 104. In the current system 10, each chamber 102, 104 has a plenum 36, 38 that supplies the chamber 102, 104 with heated dry air for sterilization. In other sterilization systems the same airflow path is used for each sterilization chamber, which may cause cold spots in the chambers and often results in one chamber reaching a higher temperature more quickly than the other chamber. The present sterilization system 10, however, uses two separate continuous airflow loops to achieve more uniform heating in each chamber 102, 104. This ultimately results in shorter batch cycle times and increased heating efficiency.

The sterilization system 10 is capable of sterilizing at least two containers in one batch cycle wherein each container is loaded into the sterilization chambers 102, 104 of the system 10. In an embodiment according to the present invention, the containers may be loaded into the chambers 102, 104 side by side.

The safety airflow switches (not shown) are sensors that can shut down the heaters 28 (FIG. 2) if they sense that there is no air flowing over the heater 28. The safety airflow switches are pressure differential switches; they measure airflow of the air intake and the air outlet. For example, if the exhaust blower 4 (FIG. 3) is not functioning and/or the recirculating fan 1 (FIG. 4) is not functioning and the air is not moving, the safety airflow switches will shut down the heaters 28 to prevent the system 10 from overheating.

In operation, there are three segments to the sterilization process: ramping, heating and cooling. In the ramping process, the chambers 102, 104 will increase heat from ambient temperature to a set point temperature, which is typically about 300° F. The length of time it takes to ramp the temperature to 300° F. depends on the load in the chambers 102, 104 and the ambient temperature in the system. Typically, the target time is between 30 to 40 minutes. The recirculating fan 1 (FIG. 4) is sized and configured to deliver a predetermined volume air to the sterilizing process at a static pressure in the range of 0.5° water column.

Typically, the user will run test to determine the time it takes to heat up the first and second chambers 102, 104 before the actual sterilization process. The user will locate the coolest spot in the chambers 102, 104 and place a thermocouple in that spot and determine the time it takes the coolest spot to reach the desired temperature.

Then, there is a pre-determined soak period at the set point temperature. During this period, hot air will circulate within the system 10 and sterilize the content for the predetermined time period. In an embodiment according to the present invention, the soak period may be approximately 30 minutes.

When the cycle is over, the sterilization system 10 will cool down the system as rapidly as possible. This is when the intake and exhaust volume increase to try to extract the heat out of the box. In the cooling process, the system 10 will cool down to about 140° F. At that temperature, operators can safely handle the load in the first and second chambers 102, 104 without getting burned. During this time, the heaters 28 are turned off and the exhaust capacity is increased while recirculating fan 1 continues to operate.

In the present embodiment, the housing 100 and the containers used are made with stainless steel, or other suitable metals to be used for a dry heat sterilization system. Furthermore, this sterilization system 10 can be used to sterilize animal cages that are used for housing rodents, canines, poultries, and mammals. In the alternative, the sterilization system 10 may also be used for sterilizing biological or chemical contaminants.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

While the disclosure has been described in terms of exemplary embodiments, those skilled in the art will recognize that the disclosure can be practiced with modifications in the spirit and scope of the appended claims. These examples given above are merely illustrative and are not meant to be an exhaustive list of all possible designs, embodiments, applications or modifications of the disclosure.

What is claimed is:

1. A sterilization system comprising:
an air intake and an air exhaust fluidly connected to a conditioning plenum;
a heater arranged inside the conditioning plenum downstream from the air intake;
a first sterilization chamber adjacent to a second sterilization chamber;
a first pressure plenum adjacent to the first sterilization chamber and fluidly connected to the first sterilization chamber and the conditioning plenum;
a second pressure plenum adjacent to the second sterilization chamber and fluidly connected to the second sterilization chamber and the conditioning plenum;
a first return duct arranged between the first and second sterilization chambers and fluidly connected to the first sterilization chamber and the conditioning plenum; and
a second return duct arranged between the first and second sterilization chambers and fluidly connected to the second sterilization chamber and the conditioning plenum.

2. The sterilization system of claim 1, further comprising a housing wherein the first sterilization chamber, the second sterilization chamber and the conditioning plenum are contained within the housing.

3. The sterilization system of claim 1, further comprising an air intake filter box coupled to the air intake.

4. The sterilization system of claim 1, further comprising an air exhaust filter box coupled to the air exhaust.

5. The sterilization system of claim 4, further comprising an exhaust blower coupled to the exhaust filter box wherein the exhaust air blower directs air to flow from the conditioning plenum into the exhaust filter box and through the air exhaust.

6. The sterilization system of claim 1, further comprising at least one deflector mounted inside the conditioning plenum.

7. The sterilization system of claim 1, further comprising semi-pierced duct walls mounted inside the first and second sterilization chambers such that the first and second pressure plenums penetrate through the semi-pierced duct walls.

8. The sterilization system of claim 7, wherein the semi-pierced duct walls further comprise adjustable diffuser panels.

9. The sterilization system of claim 1, further comprising a recirculating fan arranged inside the conditioning plenum downstream from the heater to recirculate air.

10. The sterilization system of claim 1, wherein heated air flows from the conditioning plenum to the first pressure plenum and to second pressure plenum.

11. The sterilization system of claim 10, wherein air flows through the first sterilization chamber in a horizontal direction across the first sterilization chamber.

12. The sterilization system of claim 10, wherein air flows through the second sterilization chamber in a horizontal direction across the second sterilization chamber.

13. A method of sterilizing animal cages comprising the steps of:
(a) providing a sterilization system including an air intake and an air exhaust fluidly connected to a conditioning plenum; a heater arranged inside the conditioning plenum downstream from the air intake; a first sterilization chamber adjacent to a second sterilization chamber; a first pressure plenum adjacent to the first sterilization chamber and fluidly connected to the first sterilization chamber and the conditioning plenum; a second pressure plenum adjacent to the second sterilization chamber and fluidly connected to the second sterilization chamber and the conditioning plenum; a first return duct arranged between the first and second sterilization chambers and fluidly connected to the first sterilization chamber and the conditioning plenum; and a second return duct arranged between the first and second sterilization chambers and fluidly connected to the second sterilization chamber and the conditioning plenum;
(b) placing animal cages to be sterilized in the first and second sterilization chambers;
(c) directing heated air from the conditioning plenum to the first pressure plenum and from the first pressure plenum to the first sterilization chamber and from the first sterilization chamber through the first return duct;
(d) directing heated air from the conditioning plenum to the second pressure plenum and from the second pressure plenum to the second sterilization chamber and from the second sterilization chamber through the second return duct;
(e) returning the air from the first return duct to the conditioning plenum and
(f) returning the air from the second return duct to the conditioning plenum.
